(19) United States
(12) Patent Application Publication
(10) Pub. No.: US 2004/0039352 A1
(43) Pub. Date: Feb. 26, 2004

(54) Nasal dosing device

(76) Inventors: Paul Bergeson, Tempe, AZ (US); John Christopher Shaw, Centerville, OH (US); Haven E. Bergeson, Salt Lake City, UT (US)

Correspondence Address: ELLIS & VENABLE, PC, 101 NORTH FIRST AVE., SUITE 1875, PHOENIX, AZ, 85003, US

(21) Appl. No.: 10/312,091

(22) PCT Filed: Jun. 28, 2002
(86) PCT No.: PCT/US02/21404

(51) Int. Cl. A61M 31/00

(52) U.S. Cl. 604/275; 604/187

(57) ABSTRACT

Disclosed is an end treatment, end structure, or end portion for a nasal dosing device. More particularly, the invention comprises a nasal insert tube for a nasal dosing device. The nasal insert tube is constructed having an outer surface with at least two levels thereby forming a hub. The nasal insert tube has a blunt dispensing end with at least one ejection port that is positioned substantially perpendicularly to the longitudinal axis of the nasal insert tube. Further, the distance from the at least one ejection port to the hub substantially matches a preferred distance from the outer opening of the nasal passage (i.e. the nares) to a location inside the nasal passage shown to augment the effectiveness of nasally administered medicine. For infants, the preferred distance is approximately 1 cm. The invention may be incorporated within in several alternate embodiments. The invention may be incorporated into a syringe tube, a gas propelled nasal device, or an adapter for either a syringe tube or a gas propelled nasal dosing device.

### FIELD OF THE INVENTION

The present invention relates to the field of nasal dosing devices. More particularly, the invention relates to field of end treatments or end portions of nasal dosing devices. The invention is particularly relevant to medical devices that are useful for human infants.

### BACKGROUND

Research has shown that the effectiveness of topical nasal decongestants in infants and children is highly dependent upon exactly where in the patient's nose the medication is placed. The placement of drops may be even more important than the choice of medication involved. Children, including infants less than six months of age have a remarkable response when nose drops are placed 1.0 cm into the nostril from the outer opening of the nasal extensive search of medical literature revealed no documentation of this fact; in fact, almost uniformly, practicing physicians have little practical knowledge about the anatomy, biomechanics, or physiology of the infantile nasal airway. For adults, while less precision is required, further research has shown major advantages pursuant to a system of a carefully placing dosages. Such careful placement permits a reduced dosage of medicine to achieve the same beneficial effects of a larger less carefully placed dosage.

Infant dosage is particularly critical. Small variations in both a medicinal volume and strength may in one case substantially decrease the medicines effectiveness or and in another case result in toxicity. Infants under approximately 6 months of age may be obligatory nasal breathers. Nasal congestion and/or obstruction may, therefore, be particularly serious in this age group. No known medical treatment for this problem has been proven to be both safe and effective. The narrowest part of the nasal airway has been termed the nasal valve and has now been studied using acoustic rhinometry. It is located about 1 cm proximal to the alar rim in newborns and is located at the junction of the compliant cartilaginous vestibule with the rigid bony cavity of the nose. The cross section area is very small (approximately 20 mm²). The existing related art, such as infant nose droppers, are either ineffective or untrustworthy for measuring exact dosages and placing such dosages at the nasal valve.

Thus, there is a need for a nasal dosing device that facilitates improved accuracy in measured dosage and the placement of the dosage for increased medical effectiveness.

### SUMMARY

It is an object of the present invention to provide an end treatment, end structure, or end portion for a nasal dosing device. More particularly, the invention comprises a nasal insert tube 2 having an outer surface with at least two level (i.e. at least two different distances between the longitudinal axis of the nasal insert tube and the outer surface) thereby forming a hub 18 (wherein the hub need not be continuous over the entire outer surface). Further the nasal insert tube 2 has a blunt dispensing end 16 with at least one ejection port 4 that is positioned substantially perpendicularly to the longitudinal axis of the nasal insert tube 2. Further, the distance (D) from the at least one ejection port 4 to the hub 18 substantially matches a preferred distance from the outer opening of the nasal passage (i.e. the nares) to a location inside the nasal passage shown to augment the effectiveness of nasally administered medicine. For infants, the preferred distance is approximately 1 cm. The invention may be incorporated into many alternate embodiments, the description of which are not intended to narrow the scope of the claims. Alternate examples embodying the invention include those devices for using force to propel the dose into a nasal cavity. Such examples include, a syringe tube, a gas propelled nasal dosing device, a mechanical or liquid displacement device, or an adapter for either a syringe tube, a gas propelled nasal dosing device, or a mechanical or liquid displacement device.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE DRAWINGS

FIG. 1a shows a side view of a nasal dosing device incorporating a preferred embodiment of the invention;

FIG. 1b shows a side view of a second nasal dosing device incorporating a preferred embodiment of the invention;

FIG. 2a shows a side view of a preferred embodiment of the present invention;

FIG. 2b shows a side view of a cutaway of a preferred embodiment of the present invention, a plunger is depicted to illustrate one possible embodiment;

FIG. 2c shows a side view of a cutaway of a preferred embodiment of the present invention applicable for non-plunger type nasal dosing devices;

FIG. 3a shows a side view of an alternate preferred embodiment of the present invention;

FIG. 3b shows a side view of a cutaway of the alternate preferred embodiment of the present invention, a syringe tube is depicted to illustrate one possible use of the embodiment; and

FIG. 3c shows a side view of a cutaway of the alternate preferred embodiment of the present invention.

### DESCRIPTIONS OF PREFERRED EMBODIMENTS

Alternate embodiments incorporating the invention are described below. Each embodiment includes a nasal insert tube 2 having a hub 18 at one end of the nasal insert tube 2 and a blunt dispensing end 16 at the other end of the nasal insert tube 2. Moreover, an ejection port 4 is positioned substantially perpendicularly to the longitudinal axis of the nasal insert tube 2.

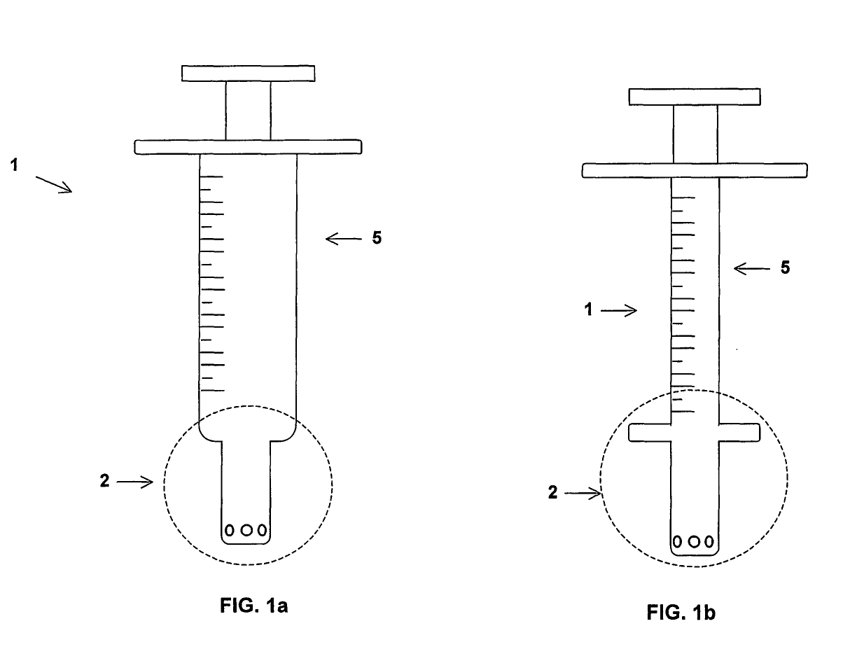

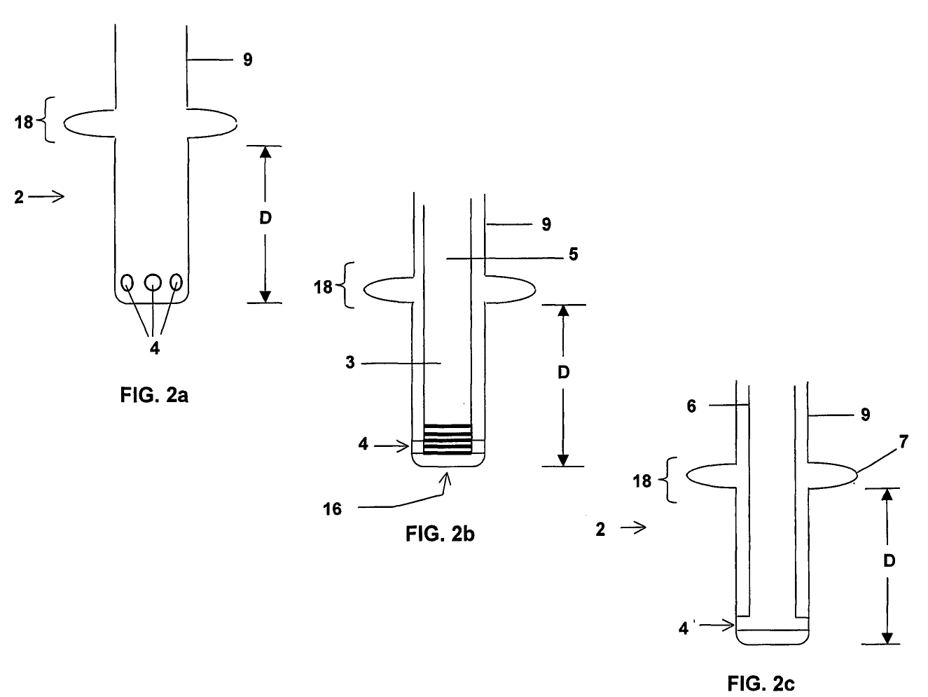

A first embodiment of a nasal dosing device incorporating the present invention is depicted in FIGS. 1a & 1b. In the first embodiment, the nasal insert tube 2 is incorporated as one end of a syringe tube 5. FIGS. 2a-2c depict more detail of the first embodiment. The preferred nasal insert tube 2 of the first embodiment is comprised of an inner annular surface 6 and an outer annular surface. The outer annular surface 8 is further comprised of a first outer annular surface portion 7 and a second outer annular surface portion 9. Moreover, the first outer annular surface portion 7 is displaced relative to the second outer annular surface portion 9 thereby forming a hub 18. In use, a plunger 3 may travel the inner annular surface 6 of the nasal insert tube 2 to propel a dose of medicine (i.e. the plunger is a medicine dosing propellant) through the ejection port 4 positioned perpendicularly to the longitudinal axis of, and substantially near the blunt dispensing end 16, of the nasal insert tube 2. Alternatively, as depicted in FIG. 2c, a non-plunger type of nasal dosing device—such as a gas propellant nasal dosing device (i.e. an alternative medicine dosing propellant)—may also incorporate the present invention. The first embodiment preferably includes a plurality of ejection ports 4 positioned as described above and no ejection ports 4 positioned in the bottom of the blunt dispensing end 16. Moreover, it is further preferable to omit any ejection ports 4 positioned substantially parallel to the longitudinal axis of the nasal syringe tube to prevent the dosing of medicine in line with the nasal passage. It is also preferable to minimize the volume of the nasal insert tube 2 cavity that cannot be displaced by the end of the plunger 3. See FIG. 2b.

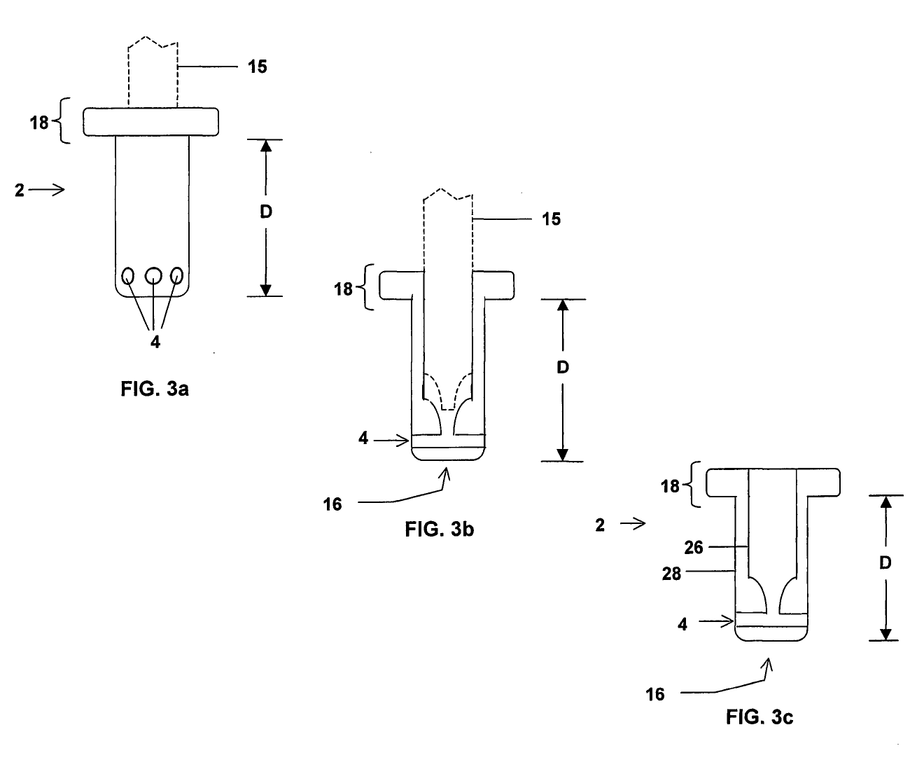

A second embodiment incorporating the invention is depicted in FIGS. 3a-3c. These figures depict an adapter for a nasal dosing device having a inner surface and an outer surface thereby forming a dosing cylinder 15 (e.g. an ordinary syringe tube or gas propelled nasal dosing device). In the second embodiment, the nasal insert tube 2 comprises an inner end portion surface 26 having a first portion that is substantially parallel with the longitudinal axis of the nasal insert tube 2 and an outer end portion surface 28 having at least two levels thereby forming a hub 18. The first portion of the inner end portion surface 26 is capable of being coupled to a dosing cylinder 15 (e.g. an ordinary syringe tube as in FIG. 3b, or the nose of a gas propelled nasal dosing device) such as by a pressure fit. The nasal insert tube 2 of the second embodiment also includes a blunt dispensing end 16 and an ejection port 4 that penetrates, and that is positioned substantially perpendicularly to the longitudinal axis of, the nasal insert tube 2. Moreover, the inner end portion surface 26 has a tapered end to match the end of the nasal dosing device and thereby increase dosing accuracy by minimizing the cavity formed between the nasal insert tube 2 and the nasal dosing device that is being adapted. See FIG. 3c. Again, it is preferred that a plurality of ejection ports 4 be incorporated into this embodiment of the invention but omitted in a position substantially parallel to the longitudinal axis of the nasal syringe tube to prevent the dosing of medicine in line with the nasal passage

The invention is used by first drawing a dose of mecdicine into the syringe tube 5 or adapted dosing cylinder 15, which are preferably calibrated and marked to accurately measure a dose. See FIG. 1. A dose of medicine may be measured by drawing the plunger 3 out of the syringe tube 5 or dosing cylinder 15 until the stopper on the plunger 3 reaches a mark on the syringe tube 5 or dosing cylinder 15 indicating a desired measured dose. Preferably, the stopper on the plunger 3 has a colored line substantially parallel to the marks on the calibrated cylinder for measuring the dose. Next, the syringe tube 5 is inserted into the nose until the hub 18 meets the alar rim of the nose. The dose is subsequently administered by forcing the plunger 3 into the syringe tube 5 or dosing cylinder 15 until the stopper of the syringe tube 5 dispenses the dose to the nose.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A nasal dosing device for a human nasal passage having an alar rim comprising:
an inner surface;
an outer surface having at least two levels wherein the at least two levels form a hub;
a nasal insert tube having a longitudinal axis and extending from the hub to a blunt dispensing end;
at least one ejection port near the blunt dispensing end and substantially perpendicularly to the longitudinal axis of the nasal insert tube; and
a medicine dosing propellant adapted to the inner surface;
wherein the nasal insert tube is adapted to the human nasal passage and the hub is adapted to contact the alar rim.

2. The nasal dosing device in claim 1 wherein,
the at least one ejection port is positioned substantially near the blunt dispensing end.

3. The nasal dosing device in claim 1 wherein,
the at least one ejection port is between 0.75 and 1.25 centimeters from the hub.

4. The nasal dosing device in claim 1 wherein,
the at least one ejection port is substantially 1 centimeter from the hub.

5. The nasal dosing device in claim 1 wherein,
the medicine dosing propellant is selected from the group consisting of a mechanical displacement device and a liquid displacement device.

6. The nasal dosing device in claim 1 wherein,
the medicine dosing propellant is selected from the group consisting of plunger and a gas propellant nasal dosing device.

7. An end portion for a nasal dosing device, the nasal dosing device having an inner surface and an outer surface that form a dosing cylinder, the end portion comprising:
a nasal insert tube having a longitudinal axis, having an inner surface end portion that is substantially parallel with a longitudinal axis of the nasal insert tube and that is coupled to the nasal dosing device inner surface, and an outer surface end portion having at least two levels thereby forming a hub, the nasal insert tube further having a blunt dispensing end and at least one ejection port that is positioned substantially perpendicularly to the longitudinal axis of the nasal insert tube.

8. The end portion in claim 8 wherein,
the nasal insert tube is an adapter for nasal dosing device selected from the group of nasal dosing devices consisting of: a syringe tube, a gas propelled nasal dosing device, a mechanical displacement device and a liquid displacement device.

9. The end portion in claim 8 wherein,
the at least one ejection port is between 0.75 and 1.25 centimeters from the hub.

10. The end portion in claim 8 wherein,
the at least one ejection port is substantially 1 centimeter from the hub.

11. The end portion in claim 8 wherein,
the nasal insert tube has annular shaped surfaces.

12. A nasal dosing device for an infant nasal passage having an alar rim comprising:
a syringe tube having an inner surface and an outer surface comprising at least two levels wherein the at least two levels form a hub;
a nasal insert tube having a longitudinal axis and extending from the hub to a blunt dispensing end;
at least one ejection port near the blunt dispensing end and substantially perpendicularly to the longitudinal axis of the nasal insert tube; and
a plunger adapted to the inner surface; and
wherein the nasal insert tube is adapted to the infant nasal passage and the hub is adapted to contact the alar rim.

13. The infant nasal dosing device in claim 12 wherein,
the at least one ejection port is positioned substantially near the blunt dispensing end.

14. The infant nasal dosing device in claim 13 wherein,
the at least one ejection port is between 0.75 and 1.25 centimeters from the hub.

15. The infant nasal dosing device in claim 13 wherein,
the at least one ejection port is substantially 1 centimeter from the hub.

16. The infant nasal dosing device in claim 15 wherein,
the nasal insert tube has annular shaped surfaces.

17. A nasal dosing device for an infant nasal passage having an alar rim comprising:
a gas propellant nasal dosing device having an inner surface and an outer surface comprising at least two levels wherein the at least two levels form a hub;
a nasal insert tube having a longitudinal axis and extending from the hub to a blunt dispensing end;
at least one ejection port near the blunt dispensing end and substantially perpendicularly to the longitudinal axis of the nasal insert tube; and
a gas propellant coupled to the inner surface; and
wherein the nasal insert tube is adapted to the infant nasal passage and the hub is adapted to contact the alar rim.

18. The infant nasal dosing device in claim 17 wherein,
the at least one ejection port is positioned substantially near the blunt dispensing end.

19. The infant nasal dosing device in claim 18 wherein,
the at least one ejection port is between 0.75 and 1.25 centimeters from the hub.

20. The infant nasal dosing device in claim 18 wherein,
the at least one ejection port is substantially 1 centimeter from the hub.

21. The infant nasal dosing device in claim 20 wherein,
the nasal insert tube has annular shaped surfaces.
